(19) United States
(12) Patent Application Publication
(10) Pub. No.: US 2026/0130881 A1
(43) Pub. Date: May 14, 2026

(54) SUSTAINED RELEASE DEVICE CONTENTS FOR TREATMENT OF PARKINSON'S DISEASE AND OTHER DISORDERS

(71) Applicant: khalafalla bushara, Eden Prairie, MN (US)

(72) Inventor: khalafalla bushara, Eden Prairie, MN (US)

(21) Appl. No.: 19/388,078

(22) Filed: Nov. 13, 2025

Related U.S. Application Data
(60) Provisional application No. 63/720,215, filed on Nov. 14, 2024.

(51) Int. Cl. A61K 31/198; A61K 9/00; A61K 31/165; A61K 31/277; A61K 47/22; A61K 47/46

(52) U.S. Cl. CPC A61K 31/198; A61K 9/0053; A61K 9/009; A61K 31/165; A61K 31/277; A61K 47/22; A61K 47/46

(57) ABSTRACT

Provided here within is a saliva-activated pharmaceutical composition for use in an intra-oral sustained-release sachet. The formulation includes acacia powder in defined acacia-to-levodopa ratios effective to increase levodopa solubility by at least 300% at 25° C., maintain a localized pH of 3-5, and suppress levodopa oxidation during and after intra-oral use. In combination with levodopa, carbidopa, ascorbic acid, and optional palatability or swallowing-stimulation agents, the acacia forms a hydrocolloid matrix that stabilizes drug particles, enhances dispersion, and enables sustained, uniform release for several hours. The composition further supports consistent distribution of flavoring agents and prolonged intra-oral retention with improved sensory persistence. This acacia-dependent hydrocolloid system provides controlled salivary dissolution, biochemical stabilization, and predictable sustained delivery of levodopa/carbidopa, achieving performance not obtained with prior acacia-based formulations or with the parent sachet device.

### CROSS-REFERENCE TO RELATED APPLICATIONS

This application is a continuation of U.S. Pat. No. 12,161,755B2 and claims the benefit of priority to U.S. Provisional Patent Application No. 63/720,215, Nov. 14, 2024

### FIELD OF THE INVENTION

The present invention relates to preparation and composition of an intra-oral device for sustained release of active pharmaceutical agents including agents with poor water solubility.

### BACKGROUND

The symptoms of many disorders can be effectively ameliorated by taking medications. However, the blood levels of such medications can fluctuate between peaks and troughs if given orally, leading to variable control of symptoms. An important example of this problem is in the treatment neurological disorders, such as Parkinson's disease, in which the benefit from orally administered medications is often short lived, leading to frequent dosing, which can be inconvenient and negatively affects compliance and quality of life. Sustained release formulations taken orally (such as Sinemet products) including gastric retentive methods only have limited success (Verhagen et al., Mov Disord., 30(9), 1222-8 (2015)). Systems delivering levodopa infusion via a gastric tube have been introduced (Eggert et al., Clin Neuropharmacol., 31(3), 151-66 (2008); Nyholm et al., Neurology, 64(2), 216-23 (2005)). However, these are invasive methods with multiple risks and side effects. Subcutaneous infusions of carbidopa (CD) and levodopa (LD) were also developed to achieve sustained and continuous drug delivery for Parkinson's disease, overcoming the poor and erratic absorption of oral forms. These infusions, such as Vyalev™, use highly soluble phosphate prodrugs (foslevodopa and foscarbidopa). The phosphate modification does increase solubility by more than 100-fold compared to native LD (<1 mg/mL) and CD (<0.004 mg/mL), though enzymatic dephosphorylation is required and phosphate buffering may limit formulation flexibility (Fabbrini 2010; Krisai 2020). Earlier efforts to enhance solubility included methyl ester prodrugs like melevodopa, which achieved >250 mg/mL solubility but had chemical instability and required hydrolysis before absorption (LeWitt 2015). Physical approaches such as micronization and spray drying (with added 1,2-dipalmitoyl-sn-glycero-3-phosphocholine (DPPC); the naturally occurring surfactant in the lungs) in inhaled levodopa preparations were also used to increase the apparent solubility by forming amorphous or fine particles (Vehring 2008; Liu 2022). The inhaled forms of levodopa ((Inbrija®) is used as a rescue medication but does not provide a sustained effect. Acidifiers and antioxidants like vitamin C have also been tested to improve dissolution but do not reliably prevent CD degradation (Buchwald 2005).

Liquid formulations of levodopa-carbidopa-ascorbic acid (LCAS, or “liquid gold”) were developed to enhance absorption and reduce gastric variability in Parkinson's disease; however, clinical experience has revealed several shortcomings. Despite improved onset and tolerability in patients with delayed gastric emptying, LCAS use is limited by complex preparation, frequent hourly dosing, and poor long-term adherence. Studies have reported high incidence of dyskinesia and hyponatremia due to excessive fluid intake. The preparation is unstable due to the oxidation of LD to dopachrome and polymerized byproducts. It requires fresh daily preparation, storage in a light-protected container and refrigerated if used beyond a few hours. LCAS is a heterogeneous mixture, not a fully dissolved solution. Hence, the preparation must be shaken before each dose as the drug distribution is nonuniform.

Therefore, there remains a critical need for convenient methods and devices for orally delivering sustained amounts of medications with an acceptable side effect profile to control the symptoms. We have disclosed a device that consists of sachets containing CD/LD and ascorbic acid that can be placed in the mouth of a human subject (U.S. Pat. No. 12,161,755B2, United States). Like LCAS, the sachet contents are expected to form a suspension within the sachet when wetted by saliva. Since the solubility of ascorbic acid in water (330 mg/mL) far exceeds that of the poorly soluble CD/LD (1 mg/mL), ascorbic acid is released first depleting the amount of ascorbic acid within the sachet before the intended dose of CD/LD is released. Furthermore, the CD/LD solubility relies on an acidic environment and is impaired with the depletion of ascorbic acid resulting a large residual of undissolved CD/LD within the sachet. The variable solubility and non-uniform distribution of active ingredients within the sachet also lead to a non-uniform dosing rate over time while using the device which is intended to be held in the mouth for several hours. The invention disclosed here resolves this issue by improving the solubility of LD and ensuring a uniform distribution of LD particles within the sachet. We have tested an exhaustive list of emulsifiers and hydrocolloids in combination with LD to determine their ability to form stable suspensions. We further studied the effect of various emulsifiers and hydrocolloids on LD solubility. Our data show that adding acacia powder to the sachet contents, at a ratio of 0.5:1 to 3:1 to LD, improves both the suspension stability (measured by viscosity over time and sedimentation rate) and increase the solubility of LD by 1.7 to 8.4 fold (4.04±3.78). Clinical and laboratory testing of sachets containing acacia powder confirmed the tolerability of this method and its efficacy in achieving plasma CD/LD levels. The more uniform distribution of flavoring and palatability inactive ingredients within the sachet such as menthol also prolonged their effect while the device is in use. This is important not only for tolerance but also for maintaining saliva production and swallowing frequency which is known to reduce sialorrhea, a common problem in PD.

### PRIOR ART

Numerous formulations have employed acacia (gum arabic) or related hydrocolloids as excipients to enhance solubility, suspension stability, or flavor uniformity in pharmaceutical and food applications. Early patents such as U.S. Pat. Nos. 3,099,602 and 3,708,594 disclosed gum acacia as a suspending and dispersing agent to obtain uniform drug suspensions, while U.S. Pat. No. 4,230,687 demonstrated its use as a polymeric encapsulant for uniform dispersion of active agents and flavors. Later developments (U.S. Pat. Nos. 5,712,310 and 7,022,352 B2) expanded this role to stabilizing water-insoluble drugs and encapsulated flavors within edible matrices. In each instance, acacia acted as a vehicle or stabilizer but not as a functional solubility enhancer within a self-contained intra-oral dosage system.

Despite these disclosures, none addressed the synergistic effect of acacia acidity (pH 3-5) in enhancing solubility and preventing oxidative degradation of levodopa/carbidopa, nor the uniform retention of flavoring agents during prolonged intra-oral dissolution using a sachet delivery device. The present invention differs by integrating acacia as both a solubility enhancer and hydrocolloidal stabilizer within an oral sachet, achieving steady levodopa release, stable flavor persistence, and consistent plasma drug levels (Table 1).

In contrast to prior formulations where acacia (gum arabic) served merely as an excipient, binder, or encapsulating agent, the present invention establishes a novel functional role for acacia as both a pH-dependent solubility enhancer and stabilizer of levodopa/carbidopa within an intra-oral drug delivery system. Unlike earlier systems limited to generic suspensions or encapsulated powders, this invention integrates acacia into an oral sachet that achieves controlled salivary dissolution and sustained release. It further demonstrates a co-functional synergy between acacia and flavoring agents, resulting in uniform dispersion and prolonged menthol sensory persistence within the same hydrocolloid matrix. Quantitatively, the formulation provides a 1.7-8.4-fold increase in levodopa solubility (minimum 300% at 25° C.), surpassing the qualitative stability improvements described in the art. Moreover, no prior patents address this approach in the context of Parkinson's disease therapy, making this the first intra-oral, acacia-based sustained-release sachet for CD/LD treatment that achieves both biochemical stabilization and patient-centric delivery uniformity.

TABLE 1
Representative Prior Art Using Acacia/Gum Arabic

| | | Role of Acacia (Gum | Dosage Form/ | Key Example/ |
|---|---|---|---|---|
| Patent No./Title | Claims Focus | Arabic) | Application | Finding |
| U.S. Pat. No. 3,099,602 - Process | Vehicle | Cold-water-dispersible | Liquid/ | Prepared stable, |
| of preparing gum acacia | formulation for | carrier improving | suspension | uniform vehicle for |
| pharmaceutical vehicle | fat-soluble | uniform dispersion | | fat-soluble drugs |
| | actives | | | |
| U.S. Pat. No. 3,708,594 - | Fine suspension | Primary suspending | Oral suspension | Gum acacia |
| Preparation containing | of poorly soluble | agent to maintain | | produced fine, |
| chloramphenicol | antibiotic | dispersion | | stable, non-settling |
| palmitate | | | | suspension |
| U.S. Pat. No. 4,230,687 - | Encapsulated | Encapsulation matrix | Microcapsules/ | Stable, evenly |
| Encapsulation of active | active agents & | for uniform | flavors | distributed |
| agents in polymeric | flavors | microdispersions | | encapsulates using |
| matrices | | | | acacia matrix |
| U.S. Pat. No. 5,712,310 - | Uniform | Hydrocolloidal | Pharmaceutical | Gum arabic |
| Suspension of water- | suspensions of | viscosity modifier/ | suspension | maintained |
| insoluble drugs | hydrophobic | stabilizer | | homogeneity & |
| | APIs | | | shelf stability |
| U.S. Pat. No. 6,193,999 B1 - | Capsule | Soluble gelatin-acacia | Soft gelatin | Improved |
| Gum acacia substituted | formulation | blend for capsule | capsule | solubility/clarity of |
| soft gelatin capsules | | shells | | shell matrix |
| U.S. Pat. No. 7,022,352 B2 - | Flavor | 30-60% acacia in | Confectionery/ | Improved flavor |
| Encapsulated flavors | encapsulation & | matrix; enhanced | chewing gum | distribution and |
| and chewing gum using | controlled | flavor retention & | | storage stability |
| same | release | uniformity | | |
| US 2006/0165775 A1 - | Film and capsule | Enhances solubility of | Oral films/ | Increased uniformity |
| Capsule and film- | coating | other polymers, | capsules | of dosage films with |
| forming composition | | smooth film formation | | acacia |
| comprising gum arabic | | | | |

Medical literature confirms that gum acacia has been widely investigated as a natural polymer for enhancing drug solubility and stability, but none disclose its application as a pH-modulated solubility enhancer in an intra-oral sustained-release system. Patel et al. (2025) reviewed GA's role as a biocompatible emulsifier and stabilizer in diverse dosage forms—nanoparticles, hydrogels, and oral or injectable systems—where it primarily functions as a matrix material or colloidal carrier without active modulation of pH or intra-oral release control. Firdouse et al. (2025) synthesized GA-stabilized ZnO/TiO₂ nanoparticles embedding entacapone for Parkinson's therapy, achieving extended release but relying on inorganic oxide matrices and not an oral or mucosal delivery route. Likewise, Shukla et al. (2025) demonstrated acacia and other gums in solid dispersions or precipitation-inhibiting beads to improve dissolution of drugs such as etoricoxib and ketoconazole, yet these formulations remain swallowed oral solids with no salivary interaction or sensory control. In contrast, the present invention employs acacia as a dual-function hydrocolloid and acidic solubility enhancer (pH 3-5) within an oral sachet that enables controlled salivary dissolution, sustained levodopa/carbidopa release, and uniform flavor distribution. This approach delivers quantified solubility gains (1.7-8.4-fold; ≥300% at 25° C.) and stable plasma levels while preserving palatability, establishing a novel intra-oral therapeutic platform fundamentally distinct from previously reported acacia-based drug-delivery systems.

In summary, prior formulations employing acacia (gum arabic) have traditionally used the material only as a binder, suspending agent, encapsulant, or viscosity modifier in oral, food, or confectionery systems, without any teaching or suggestion that acacia could function as a pH-modulated solubility enhancer for poorly water-soluble drugs such as levodopa and carbidopa. Prior art does not disclose or predict the ability of acacia to increase levodopa solubility by ≥300%, maintain a localized pH of 3-5, reduce oxidative degradation, improve suspension stability, or sustain uniform release of flavoring agents during intra-oral residence. None of the known acacia-containing formulations address the unique challenges of in-mouth, saliva-activated sustained delivery, nor do they recognize the synergistic hydrocolloid behavior that enables prolonged palatability and uniform dosing within an intra-oral sachet. The present invention therefore introduces a previously unrecognized and non-obvious functional role for acacia in enhancing solubility, stability, and controlled release of levodopa/carbidopa within the specialized sachet device of the parent patent.

A prior patent (U.S. Pat. No. 12,161,755 B2) describes related sachet delivery devices; however, the present invention provides distinct acacia-dependent compositions, stabilization mechanisms, and functional features that are not disclosed in the earlier work. The present invention therefore represents a non-obvious departure from all cited references. It identifies, for the first time, that acacia provides a unique combination of (i) pH-mediated solubility enhancement, (ii) oxidative protection, and (iii) uniform hydrocolloid matrix formation within an intra-oral sachet-properties not taught, hinted at, or predictable from any prior art reference. providing uniform particle dispersion, sustained palatability, and controlled release of the active pharmaceutical agents during sachet intra-oral residence.

### SUMMARY OF THE INVENTION

The present invention provides a novel composition of a mixture of emulsifiers/hydrocolloids, active and inactive flavoring and palatability agents to be specifically used as contents of a drug delivery device consisting of a sachet held in the mouth. Based on experimental data, specific combinations of compounds were shown to be essential for the function of the sachet device by improving solubility, distribution uniformity and suspension stability for steady release over time of the pharmaceutical agents. In vitro and in vivo studies show that acacia, when used at defined ratios, uniquely functions as a solubility-enhancing and pH-modulating hydrocolloid within the intra-oral sachet environment, increasing levodopa solubility by at least 300%, reducing oxidation, and maintaining uniform dispersion for several hours. These properties have not been described for acacia in any prior formulations and arise specifically from its use in the saliva-activated sachet method, which enables stable and sustained intra-oral release not achievable by conventional oral dosage forms.

### BRIEF EXPLANATION OF FIGURES

FIG. 1: Solubility of LD with 50 mg, 150 mg, 300 mg of acacia (AC) added to 100 mg of LD.

FIG. 2: Average plasma levels of carbidopa and levodopa in 6 normal volunteers held a sachet in the mouth for 6 hours. Sachet contained 200 mg of levodopa, 70 mg of carbidopa, 200 mg

acacia powder, 100 mg menthol and 500 mg ascorbic acid.

FIG. 3: Effect of Acacia on Particulate Dispersion After 4 Hours in Simulated Saliva. Two tubes containing activated charcoal dispersed in simulated saliva are shown after 4 hours of undisturbed settling. The tube on the right, containing activated charcoal+acacia hydrocolloid, maintains a darker and more uniformly suspended column with a markedly thinner sediment layer, indicating improved long-term dispersion stability. The tube on the left, containing charcoal without acacia, demonstrates a lighter supernatant and a thicker, denser sediment bed, reflecting more rapid particle settling in the absence of acacia.

FIG. 4. Oxidative darkening of saliva-activated levodopa sachets after 24 hours of exposure to ambient air.

### DETAILED DESCRIPTION

The present invention builds upon the well-established function of emulsifiers and hydrocolloids in dispersing and stabilizing poorly water-soluble pharmaceutical agents and extends this principle to a novel intra-oral sachet delivery platform. Specifically, it employs acacia powder (also known as arabinogalactan, Sudan gum, gum Arabic, or acacia gum), a naturally occurring polysaccharide with the general empirical composition (C₅H₈O₄)ₙ(C₁₄H₂₃O₁₀)ₘ, where “n” and “m” represent the repeating sugar units of D-galactose and L-arabinose, respectively. Acacia's highly branched molecular architecture provides a dual functionality—acting both as a hydrocolloidal stabilizer and as a weak acidic solubilizing agent—that is uniquely advantageous for formulating carbidopa/levodopa (CD/LD) in a controlled, saliva-activated sachet system.

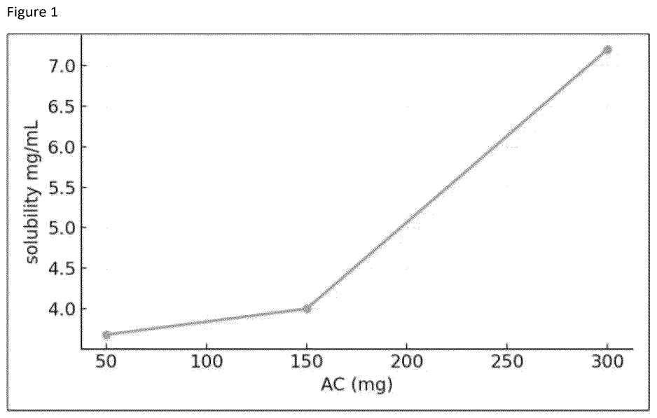

The edible, mildly sweet, and slightly acidic nature of acacia contributes to patient acceptability and chemical stability. Its intrinsic low pH (approximately 3-5) environment was found to be essential for increasing the aqueous solubility of both levodopa and carbidopa while simultaneously reducing oxidative degradation to dopachrome and other polymerized byproducts. Extensive in-vitro dissolution studies demonstrated that incorporating acacia into the sachet matrix significantly enhanced the apparent solubility of levodopa—by an average of 4.04±3.78-fold compared to control formulations—while producing a smoother dissolution curve with minimal variability over time. Optimal performance was observed within an acacia-to-levodopa ratio range of 0.5:1 to 3:1, confirming a proportional relationship between acacia concentration and solubility improvement (see FIG. 1).

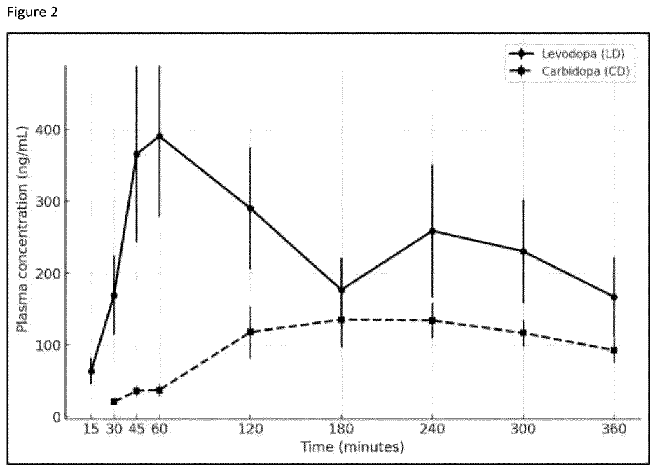

Furthermore, in-vivo pharmacokinetic testing confirmed that sachets containing acacia yielded more stable plasma concentrations of levodopa and carbidopa over the study period, indicating improved bioavailability and sustained drug release during oral retention. In addition to its physicochemical benefits, acacia facilitated a uniform distribution of flavoring agents such as menthol within the sachet matrix, producing a consistent cooling sensation and palatability that persisted throughout the six-hour evaluation (FIG. 2). Collectively, these findings establish acacia not merely as a formulation aid, but as a critical functional component enabling controlled dissolution, chemical stabilization, enhanced solubility, and sensory uniformity within a novel intra-oral sustained-release drug-delivery system for Parkinson's disease therapy.

### EMBODIMENTS OF THE INVENTION

The following embodiments further illustrate variations and implementations of the invention and are intended to supplement, without limiting, the disclosure provided herein.

In one embodiment, the intra-oral sachet composition includes acacia powder at an acacia-to-levodopa weight ratio between 0.5:1 and 3:1. Within this range, acacia forms a saliva-activated hydrocolloid matrix that increases levodopa solubility by at least 300% at 25° C., and preferably by 1.7- to 8.4-fold relative to levodopa alone. In certain embodiments, the formulation contains between 200 mg and 1200 mg of acacia, allowing modulation of viscosity, pH behavior, sedimentation stability, and the duration of intra-oral residence.

In another embodiment, the sachet contains levodopa in amounts ranging from 200 mg to 400 mg and carbidopa in amounts ranging from 70 mg to 100 mg, optionally in combination with ascorbic acid to limit oxidation and maintain a localized acidic microenvironment favorable to levodopa solubility. In some embodiments, levodopa is used in combination with benserazide or entacapone, or used alone in formulations requiring specific pharmacokinetic objectives.

Additional embodiments incorporate ascorbic acid, citric acid, tartaric acid, lemon-juice powder, or related acidifiers to stabilize levodopa, suppress oxidative degradation, and maintain an intra-sachet pH of approximately 3-5 during oral use. Ascorbic acid may be included in amounts of 1-10 wt % or in quantities between 200 mg and 500 mg, depending on the desired stability profile and the levodopa dose.

Further embodiments include palatability agents and swallowing stimulators such as menthol, mint extracts, souring agents, tingling agents, carbon-dioxide-releasing components, and umami agents. These components promote saliva flow, enhance swallowing frequency, and maintain acceptable sensory characteristics throughout prolonged intra-oral residence. In certain embodiments, menthol is present in amounts of approximately 50-100 mg to provide sustained cooling sensations for periods ranging from one to six hours.

In one embodiment, the invention includes a sachet formulation containing 400 mg levodopa, 100 mg carbidopa, 400 mg ascorbic acid, and 100 mg menthol combined with 800 mg to 1200 mg of acacia. This embodiment is suitable for high-dose delivery requiring enhanced viscosity, sustained sensory performance, and robust solubility control. In another embodiment, the sachet contains 200 mg levodopa, 300 mg ascorbic acid, and 100 mg menthol combined with 200 mg to 600 mg of acacia, providing efficient solubilization, predictable release characteristics, and balanced sensory stimulation during intra-oral residence.

In some embodiments, acacia is used alone as the principal hydrocolloid, while in other embodiments it is combined with one or more additional hydrocolloids or emulsifiers, including lecithin, carrageenan, guar gum, xanthan gum, gellan gum, gelatin, carboxymethyl cellulose, hydroxypropyl methylcellulose, casein, sodium caseinate, or sodium alginate. Such combinations may be used to further tailor viscosity, suspension stability, or dissolution kinetics to meet therapeutic or sensory requirements.

In all embodiments, saliva activation initiates formation of the hydrocolloid matrix, triggers hydration and dispersion of levodopa and carbidopa, and enables controlled, sustained release of active and sensory agents during oral retention. The extent of saliva activation may be modulated by adjusting the concentration or particle size of acacia or by selecting particular co-hydrocolloids.

Other embodiments provide compositions capable of maintaining uniform distribution of actives and sensory agents for at least four hours under typical oral conditions, thereby limiting sedimentation, preventing aggregation, and supporting consistent dosing throughout use. Such embodiments provide predictable levodopa and carbidopa release profiles and sustained palatability.

In additional embodiments, the formulation includes flavor oils, sweeteners, aromatic compounds, cooling agents, or sour agents designed to improve mouthfeel, reduce bitterness, and enhance patient comfort. The hydrocolloid matrix formed by acacia may prolong the sensory activity of these components, thereby improving adherence to therapy.

In certain embodiments, sachets may be manufactured in different strength ranges of levodopa, carbidopa, acacia, or palatability agents to allow individualized therapy, stepwise titration, or patient-specific symptom management. Embodiments may include low-dose, moderate-dose, or high-dose sachets, as well as sachets intended for sequential administration according to clinical need.

### EXAMPLES

Example 1: In a preliminary evaluation of an acacia-containing intra-oral sachet CD/LD delivery method, a single-use sachet formulation incorporating 200 mg acacia hydrocolloid, 70 mg carbidopa, 200 mg levodopa, and ascorbic acid was assessed for pharmacokinetics and tolerability. In an open-label, single-dose study with venous sampling from 15-360 minutes, six healthy adults (22-61 years) completed the assessment without significant adverse events. Levodopa demonstrated a C_max of 462.2±303.3 ng/ml, median T_max of 60 minutes, AUC_0-6 h of 1423.0±919.5 ng·h/mL, C_6 h of 167.0±136.2 ng/ml, and t½ of 225.7±117.1 minutes. Carbidopa exhibited a C_max of 150.8±88.2 ng/ml, median T_max of 210 minutes, AUC_0-6 h of 579.2±298.5 ng·h/mL, C_6 h of 92.7±44.2 ng/ml, and t½ of 347.5±268.0 minutes. These findings demonstrate that an acacia-containing sachet can achieve measurable and sustained systemic exposure to CD/LD and support further development of the intra-oral sachet platform. (FIG. 2)

Example 2: In a set of in vitro experiments, acacia-containing intra-oral sachets were evaluated for solubility and release performance using a 10-mL saliva-replacement model at 37° C. Sachets containing 300 mg acacia, 200 mg levodopa, and 500 mg carbidopa were sampled hourly and analyzed by multi-wavelength UV-Vis deconvolution. Under neutral saliva conditions with measured pH of hydrated acacia-LD mixtures between 3-5; dissolved levodopa reached ˜5 mg/mL at 60 minutes and 12-18 mg/mL at 120 minutes, with declining levels thereafter. These results demonstrate measurable, time-dependent release of levodopa from the acacia-based sachet formulation.

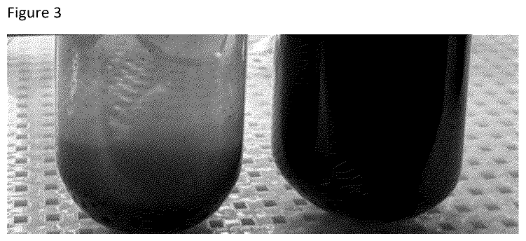

Example 3: In an in vitro study, a dispersion experiment was conducted in simulated saliva using activated charcoal as a model insoluble particulate. Tubes containing 1 g charcoal alone were compared with tubes containing 1 g charcoal plus 1 g hydrocolloid formulation. Image-based analysis showed that tubes containing the hydrocolloid exhibited a thinner sediment layer, clearer supernatant, and fewer suspended particulates, indicating reduced settling and improved dispersion relative to charcoal alone. These findings support that hydrocolloid- and acacia-containing sachet formulations enhance particulate stabilization in saliva-like environments and are consistent with their intended function in intra-oral sustained-release systems. (FIG. 3).

Example 4: In a preliminary evaluation of an acacia-containing intra-oral sachet CD/LD delivery method, a single-use sachet formulation containing 200 mg acacia hydrocolloid, 100 mg menthol, 70 mg carbidopa, 200 mg levodopa, and ascorbic acid was assessed for its effect on swallowing behavior. Swallowing events were monitored using remote Bluetooth-based sensors during sachet use and compared to each subject's baseline rate. The acacia- and menthol-containing sachet was associated with a measurable increase in swallowing frequency, indicating activation of oral sensory pathways consistent with the intended design of the formulation.

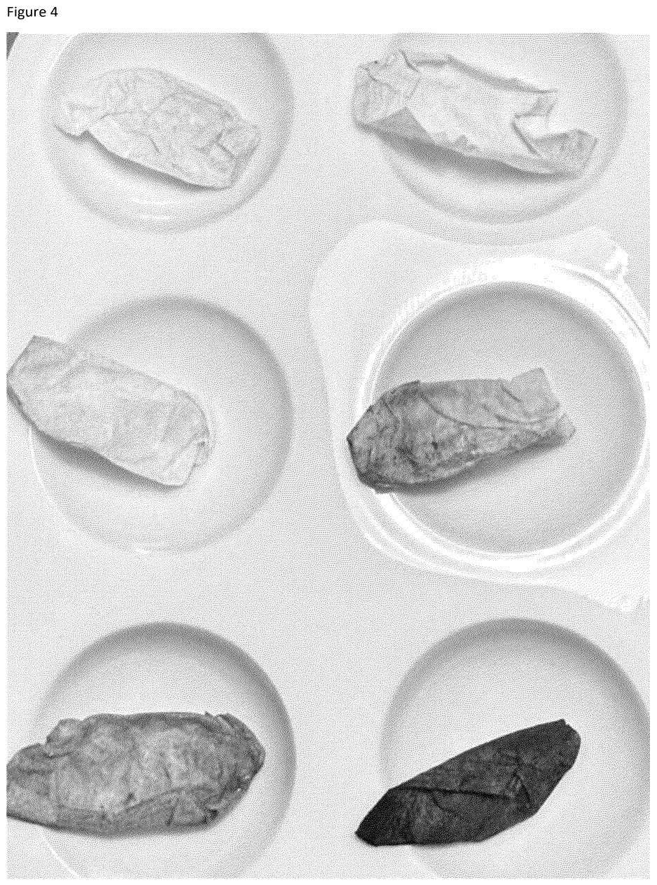

Example 5: In an experiment, saliva-activated sachets containing fixed ascorbic acid but varying acacia levels were exposed to ambient air for 24 hours. Sachets with little or no acacia showed extensive oxidative darkening of residual levodopa, whereas high-acacia sachets exhibited minimal color change. These findings indicate that acacia markedly suppresses post-activation levodopa oxidation. FIG. 4.

Provided here within is a saliva-activated pharmaceutical composition for use in an intra-oral sustained-release sachet. The formulation includes acacia powder in defined acacia-to-levodopa ratios effective to increase levodopa solubility by at least 300% at 25° C., maintain a localized pH of 3-5, and suppress levodopa oxidation during and after intra-oral use. In combination with levodopa, carbidopa, ascorbic acid, and optional palatability or swallowing-stimulation agents, the acacia forms a hydrocolloid matrix that stabilizes drug particles, enhances dispersion, and enables sustained, uniform release for several hours. The composition further supports consistent distribution of flavoring agents and prolonged intra-oral retention with improved sensory persistence. This acacia-dependent hydrocolloid system provides controlled salivary dissolution, biochemical stabilization, and predictable sustained delivery of levodopa/carbidopa, achieving performance not obtained with prior acacia-based formulations or with the parent sachet device.

### Definitions

The following definitions are provided to promote clarity and should not be interpreted as limiting unless explicitly stated otherwise. Singular terms include their plural forms and vice versa. The term “comprising” is intended to be open-ended.

### Hydrocolloid Matrix

The term “hydrocolloid matrix” refers to the hydrated, gel-like, viscous, or semi-viscous structure formed when one or more hydrocolloid components (e.g., acacia, gums, cellulose derivatives, alginates, or polymeric emulsifiers) absorb saliva and disperse within it. In the context of an intra-oral sachet, the hydrocolloid matrix provides viscosity, suspension stability, particle dispersion, pH modulation, oxidative protection, and controlled release of active and inactive agents during intra-oral residence.

### Uniform Distribution

The term “uniform distribution” refers to a state in which active pharmaceutical agents and/or sensory or flavoring agents remain evenly dispersed within the hydrated sachet contents over time. The term does not require absolute homogeneity but denotes functionally consistent dispersion without significant sedimentation, aggregation, or localized concentration gradients, thereby supporting predictable release and sensory performance during use.

### Swallowing Stimulators

The term “swallowing stimulators” refers to compounds that increase the frequency or initiation of natural swallowing reflexes through sensory, gustatory, chemesthetic, or trigeminal stimulation. Examples include sour agents (e.g., citric acid, tartaric acid), cooling agents (e.g., menthol, mint extracts), tingling or pungent agents (e.g., piperine, spilanthol, carbon-dioxide-releasing components), and umami agents (e.g., monosodium glutamate). Swallowing stimulators may help reduce sialorrhea and maintain saliva flow to support the saliva-activated release mechanism.

### Palatability Agents

The term “palatability agents” refers to substances included to enhance taste, mouthfeel, sensory acceptability, or overall user tolerability of the intra-oral formulation. Palatability agents include, but are not limited to, sweeteners, sour components, mint or cooling agents, flavor oils, aromatic compounds, and other ingredients that maintain or improve the sensory experience. The acacia-based hydrocolloid matrix may stabilize such agents and prolong their sensory effects during sachet use.

### Saliva-Activated

The term “saliva-activated” refers to formulations or components whose functional behavior—such as hydrocolloid swelling, pH modulation, dissolution, viscosity increase, dispersion of active agents, or release of flavoring or sensory compounds—is initiated or enhanced upon contact with saliva. In the disclosed sachet system, saliva activation triggers the formation of the hydrocolloid matrix and enables controlled, sustained release of levodopa, carbidopa, and associated agents during oral retention.

### REFERENCES

Buchwald H., et al. (2005). Levodopa stability and oxidation prevention in oral liquid formulations. Journal of Pharmaceutical Sciences, 94(6), 1300-1307.
Eggert K., et al. (2008). Continuous intrajejunal infusion of levodopa/carbidopa for advanced Parkinson's disease: Clinical pharmacokinetics and long-term experience. Clinical Neuropharmacology, 31(3), 151-166.
Fabbrini G., et al. (2010). Pharmacological strategies to improve levodopa therapy in Parkinson's disease. Movement Disorders, 25(Suppl 1), S60-S69.
Firdouse S., Habeeba U., Alam P., Mitta C., Kalepu S., Ahmed S. (2025). Synthesis and characterization of gum acacia-stabilized ZnO/TiO₂ nanoparticles embedded with entacapone for in vitro drug release. Journal of Research in Pharmacy, 29(1), 384-395. https://doi.org/10.12991/jrespharm.1644639
Krisai P., et al. (2020). Foslevodopa/foscarbidopa: Next-generation prodrugs for continuous subcutaneous delivery in Parkinson's disease. Drugs, 80(13), 1397-1406.
LeWitt P. A. (2015). Levodopa therapy for Parkinson's disease: Pharmacokinetics and pharmacodynamics. Movement Disorders, 30(1), 64-72.
Liu J., et al. (2022). Inhaled levodopa for Parkinson's disease: Current development and future perspectives. Frontiers in Pharmacology, 13, 873295.
Nyholm D., et al. (2005). Levodopa infusion combined with entacapone in Parkinson disease: A clinical and pharmacokinetic study. Neurology, 64(2), 216-223.
Patel R., Khumkar R., Suvarna V. (2025). Gum arabic in drug delivery systems: A route-specific overview and functional insights. Carbohydrate Polymers, 368, 124139. https://doi.org/10.1016/j.carbpol.2025.124139
Shukla A. K., Yadav V. K., Kumar P., Tiwari A., Tiwari J. (2025). Use of natural gum in poorly water-soluble drug solid dispersion and solubility enhancement. Research Journal of Pharmacy and Technology, 18(8), 77-84. https://doi.org/10.5958/0974-360X.2025.00077.1
U.S. Pat. No. 12,161,755 B2. Sustained release device for treatment of Parkinson's disease and other disorders. United States Patent and Trademark Office.
Vehring R. (2008). Pharmaceutical particle engineering via spray drying. Pharmaceutical Research, 25(5), 999-1022.
Verhagen Metman L., et al. (2015). Gastroretentive and sustained-release levodopa formulations in Parkinson's disease. Movement Disorders, 30(9), 1222-1228.

What is claimed is:

1. A pharmaceutical composition for use in an intra-oral sustained-release sachet, comprising acacia powder having the empirical structure (C₅H₈O₄)ₙ(C₁₄H₂₃O₁₀)ₘ and present at an acacia-to-levodopa weight ratio of 0.5:1 to 3:1 in an amount effective to increase levodopa solubility by at least 300% at 25° C. and maintain an acidic microenvironment within the sachet; one or more active pharmaceutical agents selected from the group consisting of levodopa, carbidopa, benserazide, entacapone, and mixtures thereof; ascorbic acid; and one or more palatability or swallowing-stimulation agents selected from citric acid, tartaric acid, lemon-juice powder, menthol, mint extracts, piperine, spilanthol, carbon dioxide, and monosodium glutamate; wherein the composition forms a saliva-activated hydrocolloid matrix that increases levodopa solubility 1.7-8.4-fold, reduces oxidation of levodopa, and maintains uniform distribution of active and palatability agents for at least four hours during intra-oral use.

2. The pharmaceutical composition of claim 1, further comprising one or more additional hydrocolloids or emulsifiers selected from lecithin, agar agar, carrageenan, guar gum, locust bean gum, polysorbate 80, sorbitan monooleate, polyglycerol polyricinoleate, sodium stearoyl lactylate, monolaurin, xanthan gum, gellan gum, gelatin, carboxymethyl cellulose, hydroxypropyl methylcellulose, casein, sodium caseinate, and sodium alginate; wherein the combined hydrocolloid system maintains suspension stability within the sachet and prevents sedimentation of the active agents for at least four hours.

3. The pharmaceutical composition of claim 1, wherein the saliva-hydrated formulation maintains suspension stability for at least four hours, the acacia hydrocolloid matrix providing uniform particle dispersion, sustained palatability, and controlled release of the active pharmaceutical agents during sachet intra-oral residence.
